(19) United States
(12) Patent Application Publication
(10) Pub. No.: US 2009/0082260 A1
(43) Pub. Date: Mar. 26, 2009

(54) COMBINATION OF AN IMMUNOSUPPRESSANT AND A PPAR GAMMA AGONIST FOR THE TREATMENT OF AN UNDESIRABLE IMMUNE RESPONSE

(76) Inventors: Jonathan Robert Lamb, Cambridgeshire (GB); Paul Kwong Tam, Pokfulam (HK)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/816,933

(22) PCT Filed: Feb. 28, 2006
(86) PCT No.: PCT/GB06/00705

§ 371 Date: Mar. 31, 2008

(30) Foreign Application Priority Data
Mar. 1, 2005 (GB) 0504206.4

(51) Int. Cl. A61K 38/13; A61K 31/421; A61K 31/426; A61P 1/00; A61P 37/06; A61P 37/00; A61K 31/4439; A61K 31/436

(52) U.S. Cl. 514/11; 514/374; 514/369; 514/342; 514/291

(57) ABSTRACT

A method for the treatment or prevention of an undesirable immune response, comprising the simultaneous administration of an immunosuppressant such as cyclosporine and a PPAR-gamma agonist, such as pioglitazone or rosiglitazone. Undesirable immune responses include, for example, rheumatoid arthritis, psoriasis, systemic lupus erythematosus, or transplant rejection.

The present invention relates to combination therapies for the treatment of undesirable immune responses, such as autoimmune disorders or transplant rejection, and compositions for use in the treatment of undesirable immune responses.

Autoimmune disorders develop when the immune system responds adversely to normal body tissues. Autoimmune disorders may result in damage to body tissues, abnormal organ growth and/or changes in organ function. The disorder may affect only one organ or tissue type or may affect multiple organs and tissues. Organs and tissues commonly affected by autoimmune disorders include blood components such as red blood cells, blood vessels, connective tissues, endocrine glands such as the thyroid or pancreas, muscles, joints and skin.

Examples of autoimmune disorders include: rheumatoid arthritis, psoriasis and lupus erythematosus. Examples of disorders which are recognised as having an autoimmune component include: inflammatory bowel disease (ulcerative colitis and Crohn's disease), Hashimoto's thyroiditis, pernicious anemia, Addison's disease, type I diabetes, systemic dermatomyositis, Sjogren's syndrome, multiple sclerosis, myasthenia gravis, Reiter's syndrome and Grave's disease. There are also disorders where the underlying mechanisms have not yet been confirmed but which involve an inflammatory component and which may or may not be autoimmune related. An example of such a possible autoimmune related disorder is atherosclerosis.

Rheumatoid arthritis (RA) is a chronic disease, mainly characterized by inflammation of the lining of the joints. It can lead to long-term joint damage, resulting in chronic pain, loss of function and disability. The disease progresses through three distinct phases. In the first stage, swelling of the synovial lining causes pain, stiffness, redness and swelling around the joint. Subsequently, in the second stage, rapid division and growth of cells causes the synovium to thicken. Followed by the third stage, where the inflamed cells lead to the breakdown of bone and cartilage, the joint then begins to lose its shape and alignment. Excessive amounts of pro-inflammatory cytokines, e.g. TNF-alpha, IL6 and IL1-beta, mediate many of the pathological features of the RA. Disease modifying antirheumatic drugs (DMARDs) and nonsteroidal anti-inflammatory drugs (NSAIDs) form the mainstay of treatment for patients with RA. Current treatment for rheumatoid arthritis is evolving towards earlier administration of DMARDs, since DMARDs may be most effective if therapy is initiated soon after disease onset, as joint destruction starts very early in the process. Recent estimates indicate that 50-70% of patients being treated for RA are administered DMARDs at some stage in their treatment, with low dose methotrexate being the most widely used DMARD due to its favourable benefit/risk profile. Other DMARDs include: hydroxychloroquine, chloroquine, gold (e.g as sodium aurothiomalate), sulfasalazine, azathioprine, mycophenolate, bromocryptine, tetracycline (and its related compounds), cyclophosphamide, D-penicillamine, bucillamine, leflunomide and corticosteroids. However, the progression of erosions continues to occur on DMARDs, although at a delayed rate compared to untreated patients. More recent therapies, such as antibodies directed against TNF-alpha (e.g. infliximab, etanercept and adalimumab) and IL6, have led to marked anti-inflammatory effects and high rates of patient response. Potent immunosuppressants such as cyclosporin A are rarely used in the treatment of RA, due to their significant adverse effects, though such medicaments may be applied in more extreme cases. For further information on therapies for RA see, for example, E. Meier et al., Elia Journal (2004) 2:7-9.

Psoriasis is a debilitating autoimmune, dermatological, disease that affects about 1-3% of the population worldwide and 2.6% of the US population (National Psoriasis Foundation, 2002). Plaque psoriasis, the most common form of the disease, is characterized by red skin covered with silvery scales. Histologically the picture is one of disordered differentiation and hyperproliferation of keratinocytes within the psoriatic plaque with inflammatory cell infiltrates (J. P. Ortonne, Brit Journal Dermatol. (1999) 140 (suppl 54) 1-7). The psoriatic skin lesions are inflammatory, red, sharply delimited plaques of various shapes with characteristic silvery lustrous scaling. The erythema, skin thickening and scaling may cover an area of up to and sometimes exceeding 50% of the body surface. It is uncomfortable, disfiguring, and not satisfactorily treated by currently available medications.

Topical treatments for psoriasis (creams and ointment formulations) include vitamin D₃ analogues (e.g calcipotriol and maxacalcitol), steroids (e.g. fluticasone propionate, betamethasone valerate and clobetasol propionate), retinoids (e.g. tazarotene), coal tar and dithranol. Topical medicaments are often used in combination with each other (e.g. a vitamin D₃ and a steroid) or with further agents such as salicylic acid.

Oral treatments for psoriasis include immunosuppressant therapies (such as methotrexate, mycophenalate and cyclosporin A) or retinoids (such as acitretin and tazarotene). Oral use of pimecrolimus is currently under investigation.

Biological agents of use in the treatment of psoriasis include anti-TNF therapies (such as monoclonal antibodies against TNF, e.g. adalimumab and infliximab, or TNF receptor fusion proteins such as etanercept), humanised antibodies to CD11a (efalizumab) or agents which bind to CD2 such as alefacept (thereby blocking the CD2 LFA3 interaction). It should be noted that not all of the biological agents listed here have been approved for use in the treatment of psoriasis.

A further treatment for psoriasis patients involved phototherapy, either simply involving UVA/UVB light or UV light in combination with psoralen therapy.

As used herein the term “psoriasis” includes psoriasis and the symptoms of psoriasis including erythema, skin thickening/elevation and scaling.

Multiple sclerosis (MS) is an inflammatory disease of the central nervous system (CNS). It is one of the most common neurological disorders affecting young adults. The specific cause(s) has yet to be identified. Local inflammation in white and grey matter leads to loss of myelin and both neuronal and axonal injury and destruction. Inflammatory factors and demyelination block or limit axonal conduction. Loss of neurons and axons is directly associated with irreversible progression of disability. There are four major clinical courses the disease to follow and each has distinct treatment responses and short-term prognoses: relapsing-remitting, primary-progressive, secondary-progressive and progressive-relapsing (F. D. Lublin et al., Neurology (1996) 46(4):907-911). However, most striking is that the clinical course and severity of symptoms can very greatly between patients depending on genetic and what are most likely to be environmental exposure factors. In general, patients with MS experience initially reversible and later progressive irreversible impairments of sensory and motor functions. Specific symptoms may include numbness, spastic muscle weakness, pain, visual problems, incontinence, loss of sexual function, speech and swallowing difficulties and loss of balance. Increasingly, cognitive impairments such as central fatigue, impairment of attention and memory and executive dysfunction are recognised as core symptoms. Symptomatic treatments (e.g., control of spasticity, limiting urinary frequency, pain control) are commonly used to modest effect at best. The only currently approved treatments to modify the course of the disease are based on immunosuppression. Methylprednisolone or related steroids may shorten the period of a relapse. Interferon-beta preparations or an immunomodulatory (proprietary) peptide mix (Cop-1) reduces the frequency of relapses and may have a small effect on rates of progression early in the disease. Substantial immunosuppression with agents such as mitoxantrone, Campath-1H or bone marrow transplantation can have a substantial impact on new inflammatory activity and probably slow rates of progression in some patients, but are unlikely to be used widely because of toxicity or associated morbidity. There is an urgent need to develop well tolerated, more selective immunosuppressants and agents that can block neurodegeneration linked to inflammation. For further information on MS see, for example, Multiple Sclerosis—National clinical guideline for diagnosis and management in primary and secondary care, The Royal College of Physicians, London, 2004 (ISBN 1 86016 182 0).

The chronic inflammatory bowel diseases, Crohn's disease (CD) and ulcerative colitis (UC) afflict both children and adults (B. A. Hendrickson et al., Clin. Microbiol. Rev. (2002) 15(1):79-94). Although IBD occurs worldwide, it is more common in the United States, United Kingdom, and Scandinavia (B. A. Hendrickson et al., Clin. Microbiol. Rev. (2002) 15(1):79-94). Incidence rates range between 4 to 10/100,000 persons per year while prevalence rates fall between 40 to 100/100,000 persons (J. B. Kirsner et al., N. Engl. J. Med. (1982) 305:837-848).

UC is defined as a condition where the inflammatory response and morphologic changes remain confined to the colon. The rectum is involved in 95% of patients. Inflammation is largely limited to the mucosa and consists of continuous involvement of variable severity with ulceration, edema, and hemorrhage along the length of the colon (B. A. Hendrickson et al., Clin. Microbiol. Rev. (2002) 15(1):79-94). UC is usually manifested by the presence of blood and mucus mixed with stool, along with lower abdominal cramping which is most severe during the passage of bowel movements. Clinically, the presence of diarrhea with blood and mucus differentiates UC from irritable bowel syndrome, in which blood is absent. Also, UC is typically diagnosed earlier than CD because the presence of blood in stool alerts the person to seek medical attention. The location of abdominal pain varies with the degree of colonic involvement.

CD can involve any part of the gastrointestinal tract from the oropharynx to the perianal area. Frequently, diseased segments are separated by intervening normal bowel (‘skip areas’), and inflammation can be transmural, often extending through to the serosa, resulting in sinus tracts or fistula formation (B. A. Hendrickson et al., Clin. Microbiol. Rev. (2002) 15(1):79-94).

Unlike UC, the presentation of CD is usually subtle, which leads to a later diagnosis. Factors such as the location, extent, and severity of involvement determine the extent of gastrointestinal symptoms. Patients who have ileocolonic involvement usually have postprandial abdominal pain, with tenderness in the right lower quadrant and an occasional inflammatory mass. Symptoms associated with gastroduodenal CD include early satiety, nausea, emesis, epigastric pain, or dysphagia. Symptoms of colonic CD may mimic UC. Finally, perianal disease is common, along with anal tags, deep anal fissures, and fistulae (B. A. Hendrickson et al., Clin. Microbiol. Rev. (2002) 15(1):79-94).

Extraintestinal features of inflammatory bowel disease (IBD) include fever, weight loss, growth failure, arthralgia, arthritis, mucocutaneous lesions such as oral aphthoid ulcers, cutaneous manifestations such as erythema nodosum and pyoderma gangrenosum (unusual: <1%), opthalmologic complications, hepatobiliary disease, primary sclerosing cholangitis (PSC), renal disease, bone abnormalities.

Several classes of therapeutic agents have utility in the treatment of both CD and UC. Sulfasalazine and the aminosalicylates (e.g. mesalazine) form the mainstays of therapy for the induction of remission in mild-to-moderately active UC and in the maintenance of remission. These agents, while not approved for the indication of CD, are also used in the treatment of mild-to-moderately active disease, but with limited utility. Adverse effects associated with these agents include nausea/vomiting and headache, as well as hypersensitivity reactions associated with the sulfa moiety of sulfasalazine.

Corticosteroids, including both prednisone and equivalent doses of other conventional steroids (e.g. budesonide), are effective in the induction of remission in active CD and UC. However, patients may experience significant adverse events (i.e. are steroid intolerant), have little or no improvement in disease activity (i.e. are steroid resistant), or flare during dose reduction or steroid withdrawal (i.e. are steroid-dependent). Conventional steroids are also ineffective at maintaining remission in either disease at doses low enough to avoid the adverse events associated with long-term use. The short-term use adverse event profile of corticosteroids includes myopathy, psychosis, glaucoma, hypertension, fluid retention, hyperglycemia, and hyperlipidemia. Long-term use is associated with osteoporosis, HPA axis suppression, cataracts, impaired wound healing, and Cushingoid appearance. CD patients treated with ileal-release budesonide have lower systemic steroid exposure and therefore a decreased likelihood of the occurrence of these side-effects, but this improved safety profile is associated with decreased efficacy.

Metronidazole is of benefit in the treatment of perianal CD for patients with mild to moderate disease, and is often used post-operatively in patients following ileal resection. Long-term use of metronidazole is limited by the risk of peripheral neuropathies.

The thiopurines azathioprine and 6-mercaptopurine are used in the maintenance of remission in both CD and UC, but have little utility in the induction of remission due to a long onset of efficacy. Toxicities associated with use of these agents include neutropenia and thrombocytopenia, hepatotoxicity, rash, opportunistic infections, and lymphoma. Overall, these agents are not universally effective, require regular toxicity monitoring, and have significant adverse event profiles. Methotrexate may also be used as a maintenance therapy in CD patients, but its use is associated with hepatotoxicity and opportunistic infections. CD and UC patients with severe refractory disease may also be treated with cyclosporin A, a therapy associated with increased risk of renal toxicity and opportunistic infections.

CD patients unresponsive to conventional therapies may be treated with a monoclonal antibody directed against the inflammatory cytokine TNF-alpha (e.g. infliximab), for both induction and maintenance of remission. Since this agent is a biologic which is delivered intravenously, side effects include infusion reactions, anaphylactoid responses, and immunogenicity. Possible increased risks of opportunistic infections accompany the use of this agent.

Atherosclerosis involves the deposition of fatty substances, cholesterol, body cellular waste products, calcium, and fibrin (a clotting material in the blood) in the inner lining of an artery. The deposited plaques can partially or totally block the flow of blood through the artery. Additionally, clot formation may occur in the region of the plaque which can also stop the flow of blood. If a blockage does occur, as a result of the plaque itself or a clot, a heart attack or stroke may result.

Atherosclerosis is a factor in several conditions including coronary heart disease (CHD), myocardial infarction (MI), angina pectoris, cerebral vascular disease (CVD), thrombotic stroke, transient ischemic attacks (TIAs), insufficient blood supply to lower limbs and feet (claudication), organ damage, and vascular complications of diabetes.

Treatments for atherosclerosis primarily include HMGCoA (3-hydroxy-3-methylglutaryl coenzyme A) reductase inhibitors, for example statins (such as atorvastatin, simvastatin and rosuvastatin, known by the brand names Lipitor, Zocor and Crestor respectively); also anti-hypertensive agents such as calcium antagonists, betablockers, ACE inhibitors and angiotensin II antagonists. There is some evidence from animal models that PPAR-gamma agonists can ameliorate atherosclerosis (C. Duval et al., TRENDS in Molecular Medicine (2002) 8(9):422-430).

Systemic lupus erythematosus (SLE) is a connective tissue auto-immune disorder, with multi-system involvement (skin, joints, kidney, nervous system, lung, heart, blood). It affects approximately 1 in 1000 women with peak of onset in 20-40 yrs and is particularly common in American-African black females (1 in 250 prevalence). Clinically the disease may be mild (skin rash/photosensitivity and arthralgia only), moderate (with additional features of inflammation of the linings of the lung and heart) and severe (seen in 15-20% patients, with involvement of major organs such as the kidney, leading to renal failure and nervous system issues). These disease states are not necessarily progressive. Fatigue is a very significant feature of all types of lupus and contributes to morbidity and inability to work even with mild disease, because of this (and limited treatment options) there is enthusiasm for new treatments for mild disease as well as for the more severe forms. The disease is characterised by flares and remissions which means there is also an interest in therapies that can be used intermittently to induce remission and then other therapies or lower doses to maintain remission.

Apart from drug-induced SLE (caused for example by isoniazid), the cause is unknown. The pathogenesis is immune complex mediated with vasculitis affecting major organs, with immune complex and complement deposition in vessels and neutrophil accumulation. Numerous auto-antibodies are associated with the disease, in particular anti-nuclear antibodies and anti-double-stranded DNA antibodies. The level of anti-DNA antibodies correlates with disease activity. Management of SLE depends on the type of the disease. However, there are no drugs with the specific indication for SLE. Skin disease is treated with anti-malarials (chloroquine or hydroxycholoroquine), joint disease with non-steroidal anti-inflammatory agents (for example ibuprofen). Moderate disease is treated with systemic steroids (e.g. prednisolone), although because of the side-effects and the long term nature of therapy, these are used sparingly. Mycophenolate (Cellcept) is also used in this group. Severe disease is treated with high dose oral steroids and cytotoxic agents mainly cyclophosphamide, possibly in combination with azathioprine and/or mycophenolate. In the most severe disease the steroids and cyclophosphamide are given as pulsed IV therapy. Cyclophosphamide has the significant side-effect of infertility, which is important due to the incidence of the disease in women of child-bearing age. The immunosuppression leads to iatrogenic immunodeficiency and severe life-threatening opportunist infections and tumours. Biological agents are not commonly used in the treatment of SLE. Anti-TNF agents have caused some concern in other patient groups due to the development of lupus-like syndromes. Anti-B-cell antibodies (anti-CD20, rituximab) appear to be effective in severe cases of the disease. Other treatments include the use of cyclosporin A, tacrolimus and thalidomide.

Organ transplantation is an ultimate option for treating end-stage organ failure. Vascularised organ transplants, including kidney, liver, heart, lung, small bowel and limb transplantation, may successfully ameliorate the existing condition, with first year graft survival of over 90%. However, clinical transplantation has not achieved its full potential as a permanent treatment for life-long diseases, with a steady 5% graft loss each year post-transplantation.

The rejection of transplanted tissue is the major barrier to the successful conclusion of a transplant procedure. Transplant rejection is the consequence of a recipient's alloimmune response to donor tissues. Rejection mechanisms may generally be characterised into three groups: hyperacute rejection, acute rejection and chronic rejection.

Hyperacute rejection occurs within the initial period following the transplant operation and is a result of the interaction of pre-existing antibodies in the recipient with donor antigens on the graft. Onset may be within minutes or hours of transplant and is characterised by thrombotic occlusions and haemorrhaging. Typically the graft will suffer irreversible damage. Hyperacute rejection is more likely to occur in individuals who have previously been exposed to non-self antigens, for example through pregnancy, blood transfusion or a prior transplant operation. The risk of hyperacute rejection may be minimised by the use of screening techniques which identify the presence of anti-graft antibodies in a potential recipient.

Acute rejection occurs within days to weeks of transplantation and is due to graft antigen recognition by T-cells. Resulting cytokine release leads to inflammation, tissue distortion, vascular insufficiency and cell destruction. The risk of acute rejection is highest in the first few months following transplantation, and may be reduced by the use of immunosuppressive agents.

Chronic rejection, which is a long term risk to transplant recipients, involves pathologic tissue remodelling. Cytokines and tissue growth factor induce smooth muscle cells to proliferate, to migrate, and to produce new matrix material. Interstitial fibroblasts are also induced to produce collagen. Histologically, progressive neointimal formation occurs within arteries and to a certain extent the veins of the graft. The resulting loss of blood flow leads to ischemia, fibrosis, and necrosis. Chronic rejection is dealt with by the long term use of a combination of corticosteroids (for example, dexamethasone, prednisolone and prednisone), immunosuppressants (such as sirolimus, tacrolimus, cyclosporin A) and antiproliferative agents (for example, methotrexate, cyclophosphamide and azathioprine). Chronic graft rejection is responsible for most late graft loss (P. Libby and J. Pober, Immunity (2001) 14(4):387-397).

Generally, the effectiveness of the immunosuppressants currently utilised in the long-term treatment of transplant recipients is tempered by their substantial side effects, both as a direct result of their immunosuppression (such as opportunistic infection and certain malignancies) and those related to the specific medication (cyclosporin A, for example, is hepatotoxic, nephrotoxic and may lead to the development of type II diabetes mellitus).

Transplant recipients will initially receive a combination of medications, typically at least a corticosteroid (which inhibits T-cell activation) and an immunosuppressant (e.g. tacrolimus or cyclosporin A). Steroid treatment is generally withdrawn as soon as possible.

While continued immunosuppressant treatment is normally required for the rest of the recipient's life to avoid chronic rejection, if significant side effects are seen as a result of the immunosuppressant treatment, the first line medicament may be replaced with a second line medication (e.g. methotrexate). It is often possible to minimise the quantities of immunosuppressant administered to a transplant recipient during maintenance therapy while ensuring that transplant rejection does not occur, however, this requires careful monitoring of the recipient to ensure that rejection does not occur.

Peroxisome Proliferator-Activated Receptor gamma (PPAR-gamma) is an orphan member of the steroid/thyroid/retinoid receptor superfamily of ligand-activated transcription factors. PPAR-gamma is one of a subfamily of closely related PPARs encoded by independent genes (C. Dreyer et. al., Cell (1992) 68:879-887; A. Schmidt et al., Mol. Endocrinol. (1992) 6:1634-1641; Y. Zhu et al., J. Biol. Chem. (1993) 268:26817-26820; S. A. Kliewer et al., Proc. Nat. Acad. Sci. USA (1994) 91:7355-7359). Three mammalian PPARs have been isolated and termed PPAR-alpha, PPAR-gamma, and NUC-1 (also known as PPAR-delta). These PPARs regulate expression of target genes by binding to DNA sequence elements, termed PPAR response elements (PPRE). To date, PPREs have been identified as the enhancers of a number of genes encoding proteins that regulate lipid metabolism, suggesting that PPARs play a pivotal role in the adipogenic signalling cascade and lipid homeostasis (H. Keller and W. Wahli, Trends Endocrin. Met (1993) 4:291-296).

European Patent 306228 describes a class of PPAR gamma agonists which are thiazolidinedione derivatives for use as insulin sensitisers in the treatment of Type II diabetes mellitus. These compounds have anti-hyperglycaemic activity. One preferred compound described therein is known by the chemical name 5-[4-[2-(N-methyl-N-(2-pyridyl)amino)ethoxy]benzyl]thiazolidine-2,4-dione and has been given the generic name rosiglitazone. Salts of this compound including the maleate salt are described in WO94/05659. European Patent Applications, Publication Numbers: 0008203, 0139421, 0032128, 0428312, 0489663, 0155845, 0257781, 0208420, 0177353, 0319189, 0332331, 0332332, 0528734, 0508740; International Patent Application, Publication Numbers 92/18501, 93/02079, 93/22445 and U.S. Pat. Nos. 5,104,888 and 5,478,852, also disclose certain thiazolidinedione PPAR-gamma agonists. Specific compounds that may be mentioned include 5-[4-[2-(5-ethyl-2-pyridyl)ethoxy]benzyl]thiazolidine-2,4-dione (also known as pioglitazone), 5-[4-[(1-methylcyclohexyl)methoxy]benzyl]thiazolidine-2,4-dione (also known as ciglitazone), 5-[[4-[(3,4-dihydro-6-hydroxy-2,5,7,8-tetramethyl-2H-1-benzopyran-2-yl)methoxy]phenyl]methyl]-2,4-thiazolidinedione (also known as troglitazone) and 5-[(2-benzyl-2,3-dihydrobenzopyran)-5-ylmethyl)thiazolidine-2,4-dione (also known as englitazone).

U.S. Pat. No. 6,294,580 (the disclosure of which is herein incorporated by reference) describes a series of PPAR gamma agonist compounds not of the thiazolidinedione class but which are instead O— and N— substituted derivatives of tyrosine which nevertheless are effective as insulin sensitisers in the treatment of Type II diabetes mellitus. One such compound has chemical name N-(2-benzoylphenyl)-O-[2-(5-methyl-2-phenyl-4-oxazolyl)ethyl]-L-tyrosine (also known as 2(S)-(2-Benzoyl-phenylamino)-3-{4-[2-5 methyl-2-phenyl-oxazol-4-yl)-ethoxy]-phenyl}-propionic acid, or by the generic name farglitazar).

U.S. Pat. No. 5,925,657 discloses methods for treating or preventing cytokine production associated with an inflammatory response by the administration of a thiazolidinedione PPAR-gamma agonist such as rosiglitazone.

U.S. Pat. No. 6,159,371 discloses methods of treating or preventing autoimmune diseases by administering an insulin resistance improving substance, such as rosiglitazone.

D. Baldwin and K. Duffin, Transplantation (2004) 77:1009-1014 demonstrate that diabetes mellitus may be safely and effectively treated following solid organ transplant through the use of rosiglitazone.

A first object of the present invention is to reduce the level of an undesired immune response to a greater extent than conventional treatments. A second object of the present invention is to enable the use of potent immunosuppressants at lower doses than conventional treatments, while maintaining a given level of immune suppression, such that the side effects experienced by patients may be reduced. A third object of the present invention is to remove the need for the prolonged treatment with potent immunosuppressants associated with conventional treatments.

According to the present invention there is provided a method for the treatment or prevention of an undesirable immune response, comprising the simultaneous administration of an immunosuppressant and a PPAR-gamma agonist.

The invention is illustrated by reference to the following figures:

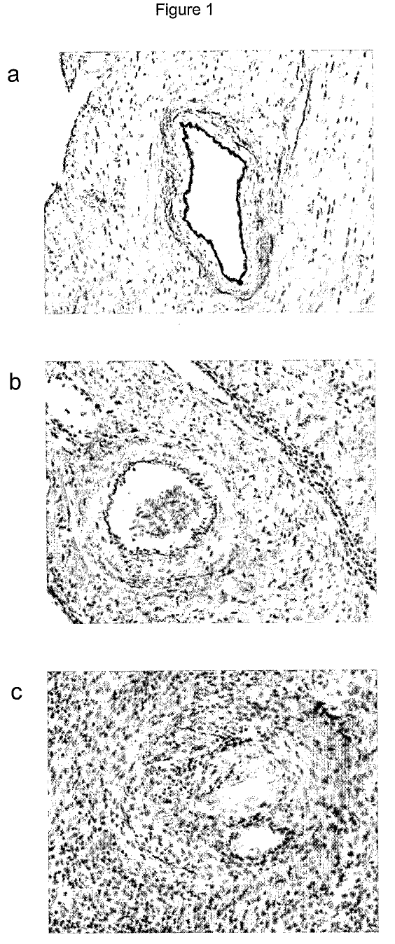

FIG. 1 shows selected sections of transplanted hearts which are representative of rejection pathology

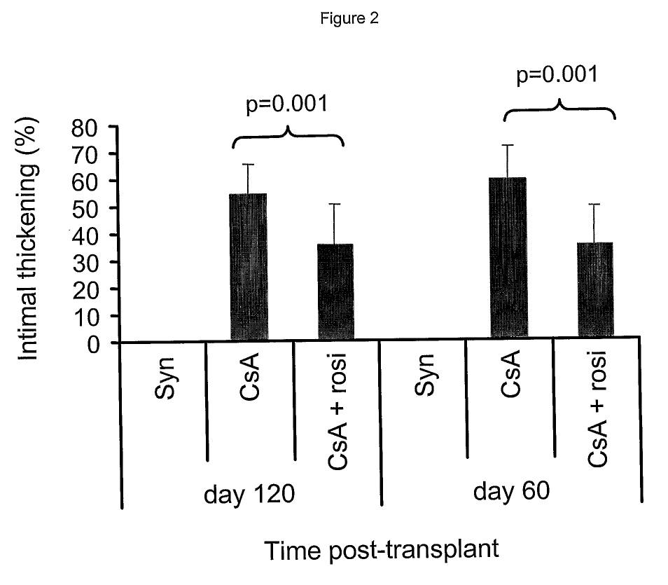

FIG. 2 compares intimal narrowing in transplant recipients undergoing a range of treatment regimes.

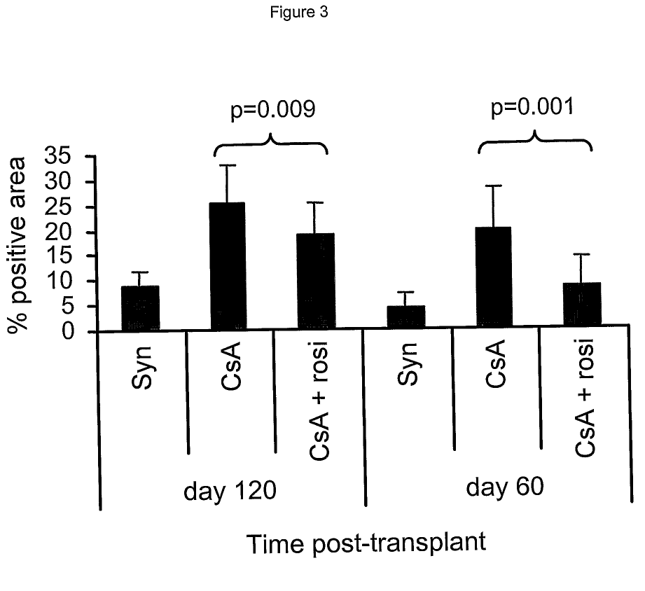

FIG. 3 compares collagen deposition in transplant recipients undergoing a range of treatment regimes.

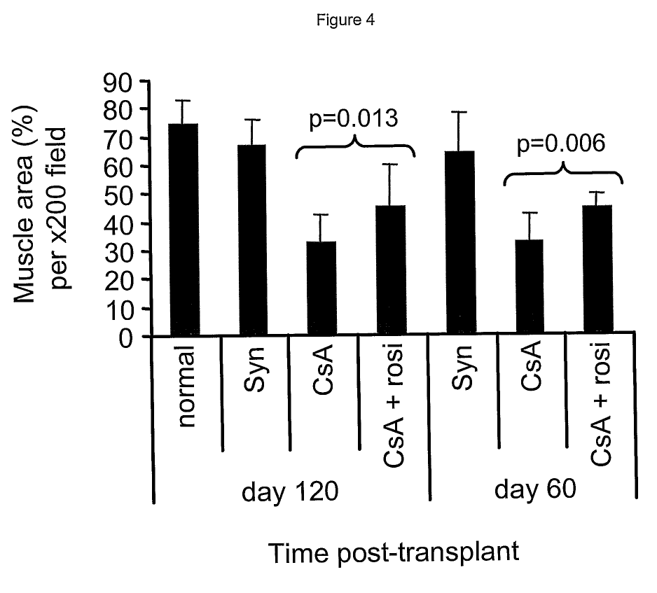

FIG. 4 compares smooth muscle area in transplant recipients undergoing a range of treatment regimes.

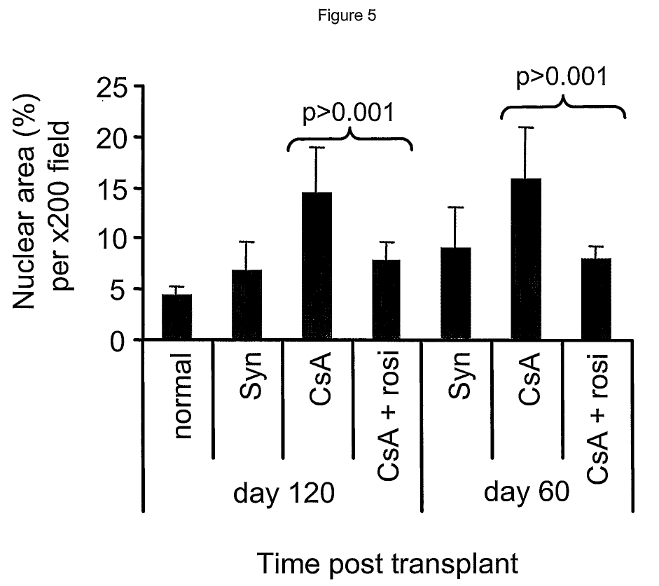

FIG. 5 compares the nuclear area in transplant recipients undergoing a range of treatment regimes.

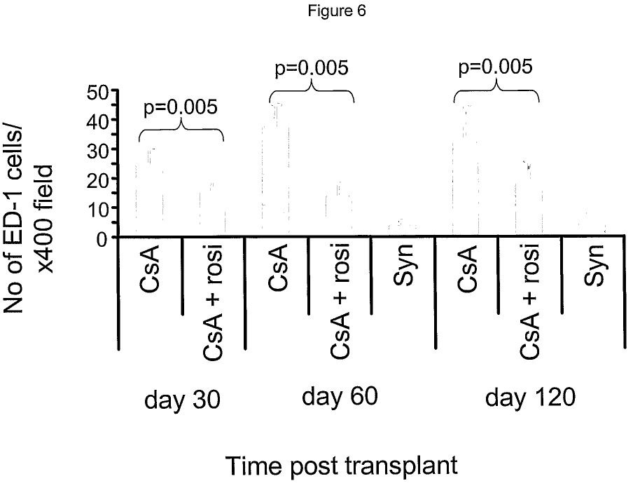

FIG. 6 compares the number of macrophages in transplant recipients undergoing a range of treatment regimes.

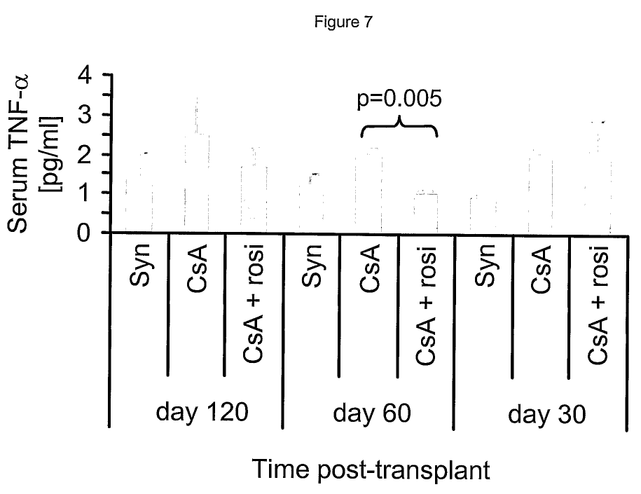

FIG. 7 compares TNF-alpha levels in transplant recipients undergoing a range of treatment regimes.

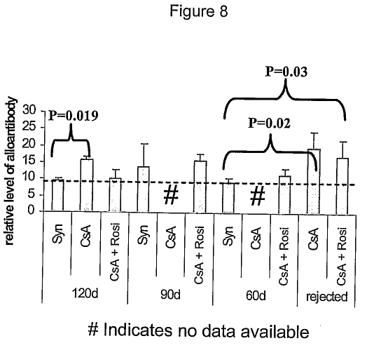

FIG. 8 compares serum IgM levels in transplant recipients undergoing a range of treatment regimes.

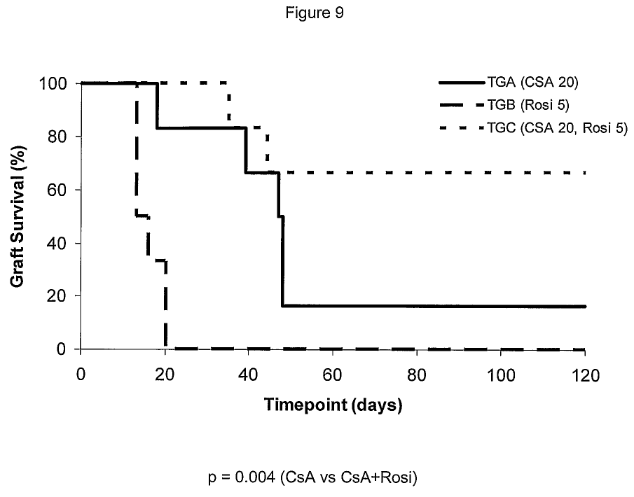

FIG. 9 compares graft survival time in transplant recipients undergoing a range of treatment regimes.

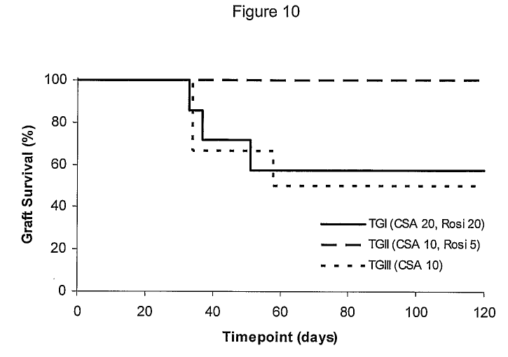

FIG. 10 compares graft survival time in transplant recipients undergoing a range of alternative treatment regimes.

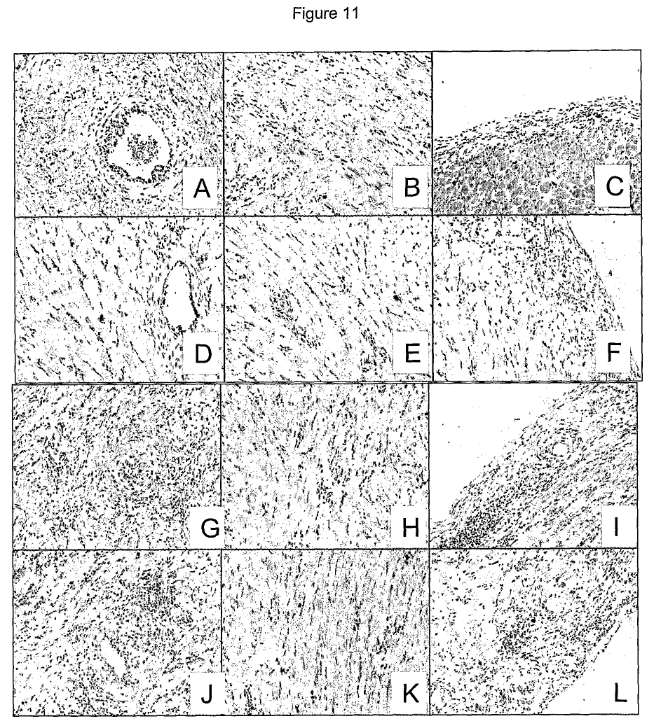

FIG. 11 compares sections from transplant recipients given primary and secondary grafts.

The undesirable immune response may arise from an autoimmune disorder, a disorder with an autoimmune component, a disorder with an inflammatory component and which may or may not be autoimmune related or a transplant operation. In one embodiment of the invention the undesirable immune response is an autoimmune disorder (for example rheumatoid arthritis, psoriasis and systemic lupus erythematosus). In a second embodiment of the invention the undesirable immune response is a disorder with an autoimmune component (for example inflammatory bowel disease (including ulcerative colitis and Crohn's disease), Hashimoto's thyroiditis, pernicious anemia, Addison's disease, type I diabetes, systemic dermatomyositis, Sjogren's syndrome, multiple sclerosis, myasthenia gravis, Reiter's syndrome and Grave's disease). In a third embodiment of the invention the undesirable immune response is a disorder with an inflammatory component and which may or may not be autoimmune related (for example atherosclerosis). In an alternative embodiment of the invention the undesirable immune response is a transplant rejection, especially either a solid organ transplant (for example kidney, liver, heart, lung, small bowel and limb) or alternatively a bone marrow transplant. In particular the undesirable immune response is one involving chronic inflammation, especially involving tissue remodelling, for example involving one or a combination of two or three of the following processes: cellular infiltration, vascular occlusion and fibrosis.

Chronic allograft rejection is mostly seen in renal grafts manifested by vascular occlusion and fibrosis of graft parenchyma. In an embodiment of the present invention the undesirable immune response is chronic renal allograft rejection. In another embodiment of the present invention the undesirable immune response is chronic cardiac allograft rejection.

The term immunosuppressant as used herein is meant to include compounds or compositions which suppress immune responses. Exemplary immunosuppressants include azathioprine, macrolides and cyclosporins, in particular macrolides (such as pimecrolimus, tacrolimus and sirolimus) and cyclosporins (such as cyclosporin A). Alternative immunosuppressants include muromonab CD3, daclizumab, basiliximab, alemtuzumab and other biological immunosuppressants (for example those targeting CD3, CD4 or CD8). In one embodiment of the invention the immunosuppressant is a macrolide, in particular tacrolimus or sirolimus, especially tacrolimus. In another embodiment of the invention the immunosuppressant is cyclosporin A. In a further embodiment of the invention the immunosuppressant is a biological immunosuppressant.

Broad spectrum immunosuppressants, such as those described in the preceding paragraph, result in a general suppression of the immune system. In one embodiment of the invention the immunosuppressant is a broad spectrum agent. However, agents which suppress only part of the immune response are also considered to fall within the scope of the present invention. In a second embodiment of the invention the immunosuppressant is one which suppresses only part of the immune response.

TNF-alpha antibodies act against the inflammatory pathway and as such may ameliorate a symptom of an immune response. In one embodiment of the invention the immunosuppressant is an TNF-alpha antibody.

Optionally, more than one immunosuppressant may be utilised in the present invention (for example, a combination of two immunosuppressants). In one embodiment of the present invention a single immunosuppressant is utilised. However, undesirable immune responses may involve multiple mechanisms and many cell types, as such, in many cases it may be the case that treatment using a combination of immunosuppressants is necessary to obtain an optimal response.

The term PPAR-gamma agonist as used herein is meant to include compounds or compositions which behave as agonists or partial agonists of the PPAR-gamma receptor. Suitable PPAR-gamma agonists of use in the present invention include docosahexaenoic acid, prostaglandin J₂, prostaglandin J₂ analogues (e.g. Δ¹²-prostaglandin J₂ and 15-deoxy-Δ^{12,14}-prostaglandin J₂), farglitazar (GI 262570), oxazolidinediones and thiazolidinediones. Exemplary thiazolidinediones include troglitazone, ciglitazone, pioglitazone, rosiglitazone (BRL 49653), darglitazone and englitazone.

Suitably, the PPAR-gamma agonist is a thiazolidinedione. In particular, the thiazolidinedione is rosiglitazone or pioglitazone, especially rosiglitazone. Farglitazar is also of particular interest.

Optionally, more than one PPAR-gamma agonist may be utilised in the present invention (for example, a combination of two PPAR-gamma agonists). Suitably, a single PPAR-gamma agonist is utilised.

If the undesirable immune response is due to the autoimmune disorder psoriasis and the PPAR-gamma agonist is rosiglitazone, suitably the immunosuppressant is not a cyclosporin (e.g. not cyclosporin A).

The term simultaneous administration as used herein in relation to the administration of medicaments refers to the administration of medicaments such that the individual medicaments are present within a subject at the same time. In addition to the concomitant administration of medicaments (via the same or alternative routes), simultaneous administration may include the administration of the medicaments (via the same or an alternative route) at different times.

Although the simultaneous administration of an immunosuppressant and a PPAR-gamma agonist may be maintained throughout a period of treatment or prevention, surprisingly, it has been found that immune suppression induced by the method of the invention may be largely maintained by subsequent administration of the PPAR-gamma agonist in isolation (for example, without the immunosuppressant of the initial phase, or alternatively without any immunosuppressant). Even more surprisingly, it has been found that immune suppression induced by the method of the invention may continue in the absence of the administration of either the immunosuppressant or the PPAR-gamma agonist.

Suitably, the subject receiving treatment for an undesirable immune response according to the present invention is not one suffering from a diabetic disorder which would typically be treated by the administration of a PPAR-gamma agonist (for example Type II diabetes mellitus, or post-transplant diabetes).

As a second aspect of the invention there is provided a method for the treatment or prevention of an undesirable immune response, comprising:
(a) an initial treatment phase comprising the simultaneous administration of an immunosuppressant and a PPAR-gamma agonist;
(b) a subsequent treatment phase comprising the administration of a PPAR-gamma agonist without the immunosuppressant of phase (a).

Further, there is provided a method for the treatment or prevention of an undesirable immune response, comprising:
(a) an initial treatment phase comprising the simultaneous administration of an immunosuppressant and a PPAR-gamma agonist;
(b) a subsequent treatment phase comprising the administration of a PPAR-gamma agonist without an immunosuppressant.

The method according to the present invention may provide an improved level of immune suppression in comparison to conventional treatments of an immunosuppressant alone. As such, it may be possible to utilise the immunosuppressant at doses which would be insufficient (i.e. sub-therapeutic) in the absence of a PPAR-gamma agonist, while maintaining the same or an adequate level of immune suppression with fewer side effects.

Where the undesired immune response is transplant rejection, treatment using a combination of an immunosuppressant and a PPAR-gamma agonist will typically begin immediately following transplantation. It may be expected that treatment according to the present invention may also be beneficial if initiated a period of time after transplantation.

In some embodiments of the invention, it may be desirable to precede treatment using a combination of an immunosuppressant and a PPAR-gamma agonist with a treatment phase using only one medicament (for example the PPAR-gamma agonist). For example, such a pre-treatment phase may be necessary where a period of time is required for the levels of one medicament to stabilise in the body.

In order to use the immunosuppressant and PPAR-gamma agonist in the invention they will normally be formulated into a pharmaceutical composition in accordance with standard pharmaceutical practice. Depending on the individual medicaments utilised, they may be formulated in combination (where a stable formulation may be prepared and where desired dosage regimes are compatible) or the medicaments may be formulated separately (for concomitant or separate administration through the same or alternative routes).

According to the present invention there is provided a pharmaceutical composition comprising an immunosuppressant and a PPAR-gamma agonist, optionally together with a pharmaceutically acceptable diluent or carrier.

Also provided is a kit of parts comprising:
(a) a pharmaceutical composition comprising an immunosuppressant;
(b) a pharmaceutical composition comprising a PPAR-gamma agonist; together with instructions for use in the treatment or prevention of an undesirable immune response.

In a further aspect of the present invention there is provided the use of a PPAR-gamma agonist in the manufacture of a medicament for the treatment or prevention of an undesirable immune response in combination with an immunosuppressant.

In another aspect of the present invention there is provided the use of an immunosuppressant in the manufacture of a medicament for the treatment or prevention of an undesirable immune response in combination with a PPAR-gamma agonist.

Also provided is the use of an immunosuppressant and a PPAR-gamma agonist in the manufacture of a medicament for the treatment or prevention of an undesirable immune response.

There is further provided a PPAR-gamma agonist for use in the treatment or prevention of an undesirable immune response in combination with an immunosuppressant.

There is also provided an immunosuppressant for use in the treatment or prevention of an undesirable immune response in combination with a PPAR-gamma agonist.

It will be clear to those skilled in the art that the medicaments may be presented in the form of pharmaceutically acceptable salts or solvates.

Suitable solvates include hydrates.

Suitable salts include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonic or naphthalenedisulphonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4 methoxybenzoic, 2- or 4-hydroxybenzoic, 4-chlorobenzoic, 4-phenylbenzoic, benzeneacrylic (for example 1,4-benzenediacrylic) and isethionic acids. Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

Where the PPAR-gamma agonist is rosiglitazone, suitably the rosiglitazone is in the form of rosiglitazone maleate. Where the PPAR-gamma agonist is pioglitazone, suitably the pioglitazone is in the form of pioglitazone hydrochloride. Where the PPAR-gamma agonist is farglitazar, an exemplary salt form is the sodium salt.

Suitable formulations include those for oral, parenteral (including subcutaneous, intradermal, intramuscular, intravenous and intraarticular), inhalation (including fine particle dusts or mists which may be generated by means of various types of metered dose pressurised aerosols, nebulisers or insufflators), rectal and topical (including dermal, buccal, sublingual and intraocular) administration, although the most suitable route may depend upon for example the condition of the recipient and the medicament in question. The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g. lactose or starch). Suitably, lactose is used.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants e.g. oleic acid or lecithin and cosolvents e.g. ethanol. Pressurised formulations will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 um, in particular 2-5 um. Particles having a size above 20 um are generally too large when inhaled to reach the small airways. To achieve these particle sizes the particles of the active ingredient as produced may be size reduced by conventional means e.g. by micronisation. The desired fraction may be separated out by air classification or sieving. Suitably, the particles will be crystalline. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 um and not less than 15% will have a MMD of less than 15 um.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Formulations for rectal administration may be presented as a suppository with the usual carriers such as cocoa butter or polyethylene glycol.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose an acacia.

It should be understood that in addition to the ingredients particularly mentioned above, the formulations of this invention may include other agents conventional in the art having regard to the type of formulation in question, for example those suitable for oral administration may include flavouring agents.

Where the PPAR-gamma agonist is rosiglitazone or pioglitazone, the compounds are suitably formulated for oral administration, in particular as a tablet. Where the immunosuppressant is tacrolimus, the compound is preferably formulated for parenteral or oral administration, especially oral administration. Where the immunosuppressant is sirolimus, the compound is preferably formulated for oral administration. Where the immunosuppressant is cyclosporin A, the compound is suitably formulated for parenteral or oral administration, especially oral administration.

Rosiglitazone is available in the form of rosiglitazone maleate (Avandia) from GlaxoSmithKline, formulated as 2, 4 or 8 mg oral tablets.

Pioglitazone is available in the form of pioglitazone hydrochloride (Actos) from Takeda Pharmaceuticals, formulated as 15, 30 or 45 mg oral tablets.

Sirolimus (Rapamycin, Rapamune) is available from Wyeth Pharmaceuticals formulated for oral administration as 1, 2, or 5 mg tablets or 1 mg/ml solution.

Tacrolimus (Prograf) is available from Fujisawa Healthcare formulated for oral administration as 0.5, 1, or 5 mg capsules or for injection as 5 mg/ml solution.

Cyclosporin A is available from a number of suppliers, for example Novartis (Sandimmune, Neoral), formulated for oral administration (25, 50 and 100 mg capsules, or 100 mg/ml solution) or for injection (50 mg/ml solution).

The method and pharmaceutical formulations according to the present invention may be used in combination with or include one or more other therapeutic agents of relevance to the specific undesired immune response. For example, anti-inflammatory agents (including NSAIDs such as naproxen, ibuprofen, diclofenac, indomethacin, nabumetone, piroxicam and asprin), corticosteroids, antiprolifierative agents and antibiotics. Suitable additional agents of use in conjunction with the present invention for the treatment or prevention of a specific undesirable immune response include those listed previously during the description of exemplary undesirable immune responses and the discussion of treatments utilised currently.

Where the immunosuppressant utilised in the invention is orally administered cyclosporin A, typical dosages will be in the order of 4-5 mg/kg twice daily. Where the immunosuppressant utilised in the invention is orally administered tacrolimus, typical dosages will begin in the order of 0.1 mg/kg/day. Where the immunosuppressant utilised in the invention is orally administered sirolimus, typical dosages will begin in the order of 2-5 mg/day. Where the PPAR-gamma agonist utilised in the invention is orally administered rosiglitazone, typical dosages will be in the order of 2-8 mg/day. Where the PPAR-gamma agonist utilised in the invention is orally administered farglitazar, typical dosages will be in the order of 2-10 mg/day.

Those skilled in the art will recognise that actual dosage levels will be determined by individual requirements and may vary from those described above.

The present invention is illustrated by the following non-limiting examples:

### EXAMPLES

### Example 1

### Formulation of a PPAR-Gamma Agonist

### Rosiglitazone Granular Concentrate Preparation

Table 1 shows the composition of the granular concentrate utilised in tablet formulation. The concentrate was prepared by passing approximately two thirds of the lactose monohydrate is through a suitable screen and blending with the rosiglitazone maleate. Sodium starch glycollate, hydroxypropyl methylcellulose, microcrystalline cellulose and the remaining lactose are passed through a suitable screen and added to the mixture. Blending is then continued. The resulting mixture is then wet granulated with purified water. The wet granules are then screened, dried on a fluid bed drier and the dried granules are passed through a further screen and finally homogenised.

TABLE 1
Composition of granular concentrate

| Ingredient | Quantity (%) |
|---|---|
| Milled rosiglitazone maleate | 13.25 (pure maleate salt) |
| Sodium Starch Glycollate | 5.00 |
| Hydoxypropyl Methylcellulose 2910 | 5.00 |
| Microcrystalline Cellulose | 20.0 |
| Lactose Monohydrate, regular grade | to 100 |
| Purified water | * |

* Removed during processing.

Formulation of the Concentrate into Tablets

Table 2, shown below, indicates the final compositions for tablets which contain 1, 2, 4 or 8 mg of rosiglitazone (based on the weight of rosiglitazone base). The tablets are prepared by first placing the granular concentrate into a tumble blender. Approximately two thirds of the lactose is screened and added to the blender. The microcrystalline cellulose, sodium starch glycollate, magnesium stearate and remaining lactose are screened and added to the blender and the mixture blended together. The resulting mix is then compressed on a rotary tablet press to a target weight of 150 mg for the 1, 2 and 4 mg tablets and to a target weight of 300 mg for the 8 mg tablets.

The tablet cores are then transferred to a tablet coating machine, pre-warmed with warm air (approximately 65° C.), where they are film coated until the tablet weight has increased by 2.0% to 3.5%.

TABLE 2
Composition of formulated tablets

| Tablet Strength | Quantity (mg per tablet) | | | |
|---|---|---|---|---|
| (mg rosiglitazone base) | 1.0 mg | 2.0 mg | 4.0 mg | 8.0 mg |
| Active Ingredient: | | | | |
| Rosiglitazone maleate granular | 10.00 | 20.00 | 40.00 | 80.00 |
| concentrate | | | | |
| Other Ingredients: | | | | |
| Sodium Starch Glycollate | 6.96 | 6.46 | 5.46 | 10.92 |
| Microcrystalline Cellulose | 27.85 | 25.85 | 21.85 | 43.70 |
| Lactose monohydrate | 104.44 | 96.94 | 81.94 | 163.88 |
| Magnesium Stearate | 0.75 | 0.75 | 0.75 | 1.50 |
| Total Weight of Tablet Core | 150.0 | 150.0 | 150.0 | 300.0 |
| Aqueous film coating material | 4.5 | 4.5 | 4.5 | 9.0 |
| Total Weight of Film Coated | 154.5 | 154.5 | 154.5 | 309.0 |
| Tablet | | | | |

### Example 2

### Cardiac Allograft Rejection in Rats (Analysis of Biological Markers)

### General

Transplant recipients were healthy male Lewis strain rats of 8-10 weeks age. Donor organs were harvested from healthy male F344 strain rats or Lewis strain rats of 8-10 weeks age.

Animals were obtained from the Animal Resources Centre, Australia. The rats were allowed free access to food and water in a 12-hour light/12-hour dark cycled room. The experimental protocol was approved by the Committee on the Use of Live Animals in Teaching and Research, University of Hong Kong.

Rats were subject to one of six experimental protocols according to the transplant methodology applied and the medicaments administered.

The PPAR-gamma agonist rosiglitazone was employed as rosiglitazone maleate (Avandia).

### Transplantation

### (i) Treatment Group 1 (TG1)—Immunosuppressant Only

Recipient rats in TG1 received hearts harvested from donor F334 strain rats. Standard methods of graft harvesting and transplant were used (M. E. Russell et al., Proc. Natl. Acad. Sci. USA (1993) 90:6086-6090).

### (ii) Treatment Group 2 (TG2)—Medium Dose PPAR-Gamma Agonist Only

Rats in TG2 received transplants following an identical procedure to that described for TG1.

(iii) Treatment Group 3 (TG3)—Immunosuppressant and Low Dose PPAR-Gamma Agonist

Rats in TG33 received transplants following an identical procedure to that described for TG1.

### (iv) Treatment Group 4 (TG4)—Immunosuppressant and Medium Dose PPAR-Gamma Agonist

Rats in TG4 received transplants following an identical procedure to that described for TG1.

### (v) Treatment Group 5 (TG5)—Syngenic Graft

Recipient rats in TG5 received hearts harvested from donor Lewis strain rats. Save for the donor rat strain, transplant methodology followed an identical procedure to that described for TG1.

### (vi) Treatment Group 6 (TG6)—Sham Transplant

Rats in TG6 were subjected to a sham transplant, wherein the abdomen of the animal was opened and closed.

### Treatment Regimes

### (i) Treatment Group 1—Immunosuppressant Only

Immunosuppressant (cyclosporin A) was administered by intraperitoneal injection at 20 mg/kg/day. Administration was started on the day of transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

### (ii) Treatment Group 2—Medium Dose PPAR-Gamma Agonist Only

PPAR-gamma agonist (rosiglitazone) was administered orally at 5 mg/kg/day. 4 mg rosiglitazone tablets were dissolved in distilled water and the rosiglitazone solution was feed to the rats once daily by gavage, in the morning. Administration was begun 3 days prior to transplantation.

(iii) Treatment Group 3—Immunosuppressant and Low Dose PPAR-Gamma Agonist

Immunosuppressant (cyclosporin A) was administered by intraperitoneal injection at 20 mg/kg/day. Administration was started on the day of transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

PPAR-gamma agonist (rosiglitazone) was administered orally at 0.5 mg/kg/day. 4 mg rosiglitazone tablets were dissolved in distilled water and the rosiglitazone solution was feed to the rats once daily by gavage, in the morning. Administration was begun 3 days prior to transplantation.

### (iv) Treatment Group 4—Immunosuppressant and Medium Dose PPAR-Gamma Agonist

Immunosuppressant (cyclosporin A) was administered by intraperitoneal injection at 20 mg/kg/day. Administration was started on the day of transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

PPAR-gamma agonist (rosiglitazone) was administered orally at 5 mg/kg/day. 4 mg rosiglitazone tablets were dissolved in distilled water and the rosiglitazone solution was feed to the rats once daily by gavage, in the morning. Administration was begun 3 days prior to transplantation.

### (v) Treatment Group 5—Syngenic Graft

Immunosuppressant and PPAR-gamma agonist were not administered to rats in TG5.

### (vi) Treatment Group 6—Sham Transplant

Immunosuppressant and PPAR-gamma agonist were not administered to rats in TG6.

### Results

### (i) Pathology

FIG. 1 shows selected sections of transplanted hearts which are representative of the rejection pathology. FIG. 1a is a section from a mouse in TG5 (a control syngenic transplant).

FIG. 1b is a section through a transplanted heart from a mouse in TG4 (cyclosporin A and medium dose rosiglitazone). FIG. 1b shows limited cellular infiltration, intimal narrowing and fibrosis.

FIG. 1c is a section through a rejected transplant taken from a mouse in Treatment Group 1 (cyclosporin A only). Significant levels of cellular infiltration and intimal narrowing have led to the vessel shown becoming occluded.

### (ii) Intimal Narrowing

The intimal thickness of TG5 (syngenic transplant) was compared to those of TG1 (cyclosporin A only) and TG4 (cyclosporin A and medium dose rosiglitazone).

Luminal (L) and intimal and luminal (I+L) distance were measured. The distance was measured with software (Adobe Photoshop). The intimal narrowing (% stenosis) was calculated according to the formula: intimal thickness=I/I+L, and expressed as a percentage increase relative to TG5 (syngenic transplant). Data is shown in Table 3 below.

TABLE 3
Intimal narrowing

| Timepoint | Treatment Group | Mean | SD | n |
|---|---|---|---|---|
| Day 120 | TG5 (syngenic transplant) | 0 | 0 | 15 |
| | TG1 (cyclosporin A only) | 54.4 | 11 | 15 |
| | TG4 (cyclosporin A and medium dose | 35.7 | 15 | 20 |
| | rosiglitazone) | | | |
| Day 60 | TG5 (syngenic transplant) | 0 | 0 | 14 |
| | TG1 (cyclosporin A only) | 59.5 | 12 | 25 |
| | TG4 (cyclosporin A and medium dose | 35.2 | 14 | 10 |
| | rosiglitazone) | | | |

n indicates the total number of vessels measured, which depended upon the number of vessels visible in the sample slides. Samples were taken from three separate animals, with one slide per animal.

FIG. 2 illustrates the data graphically.

The data were analyzed using GraphPad InStat software (available from GraphPad Software Inc, San Diego, USA) and probability values were calculated by using Tukey-Kramer Multiple Comparison Test.

Rats in TG4 (cyclosporin A and medium dose rosiglitazone) show significantly less intimal narrowing at 60 days (p=0.001) and 120 days (p=0.001) post-transplantation than those in TG1 receiving conventional treatment with cyclosporin A alone.

(iii) Fibrosis

Collagen deposition was determined for TG5 (syngenic transplant) and compared to those of TG1 (cyclosporin A only) and TG4 (cyclosporin A and medium dose rosiglitazone). The area of collagen deposition is measured by MetaMorph software (available from Molecular Devices Corporation, Downingtown, USA) on specifically stained sections (Verhoeff's Elastin). The percentage of collagen positive area was calculated against the total area.

TABLE 4
Collagen deposition

| | | | | Number of |
|---|---|---|---|---|
| Timepoint | Treatment Group | Mean | SD | x400 field |
| Day 120 | TG5 (syngenic transplant) | 9 | 3 | 30 |
| | TG1 (cyclosporin A only) | 25.48 | 7.3 | 30 |
| | TG4 (cyclosporin A and medium | 18.9 | 6.6 | 30 |
| | dose rosiglitazone) | | | |
| Day 60 | TG5 (syngenic transplant) | 4.37 | 2.94 | 30 |
| | TG1 (cyclosporin A only) | 19.78 | 8.6 | 30 |
| | TG4 (cyclosporin A and medium | 8.5 | 5.9 | 30 |
| | dose rosiglitazone) | | | |

Samples were taken from three animals at each time point (one slide per subject, with ten fields analysed from each slide).

FIG. 3 illustrates the data graphically.

The data were analyzed using GraphPad InStat software and probability values were calculated by using Tukey-Kramer Multiple Comparison Test.

Rats in TG4 (cyclosporin A and medium dose rosiglitazone) show significantly lower areas of collagen at 60 days (p=0.009) and 120 days (p=0.001) post-transplantation than those in TG1 receiving conventional treatment with cyclosporin A alone.

### (iv) Tissue Necrosis

The amount of smooth muscle cell observed in TG1 (cyclosporin A only), TG4 (cyclosporin A and medium dose rosiglitazone) and TG5 (syngenic transplant) were compared to normal heart tissue (normal being defined as tissue taken from healthy male age matched Lewis strain rats).

The area of muscle fibre was measured by MetaMorph software on specifically stained sections (Verhoeff's Elastin). The percentage of muscle area was determined relative to the total area. Higher muscle areas are considered to indicate less tissue necrosis.

TABLE 5
Smooth muscle area

| | | | | Number of |
|---|---|---|---|---|
| Timepoint | Treatment Group | Mean | SD | x200 field |
| Day 120 | Normal | 74.86 | 8.1 | 30 |
| | TG5 (syngenic transplant) | 66.89 | 8.84 | 30 |
| | TG1 (cyclosporin A only) | 32.86 | 10 | 30 |
| | TG4 (cyclosporin A and medium | 45.38 | 14.29 | 30 |
| | dose rosiglitazone) | | | |
| Day 60 | TG5 (syngenic transplant) | 64.34 | 13.78 | 30 |
| | TG1 (cyclosporin A only) | 32.86 | 10 | 30 |
| | TG4 (cyclosporin A and medium | 44.46 | 4.9 | 30 |
| | dose rosiglitazone) | | | |

Samples were taken from three animals at each data point (one slide per subject and 10 fields per slide).

FIG. 4 illustrates the data graphically.

The data were analyzed using GraphPad InStat software and probability values were calculated by using Tukey-Kramer Multiple Comparison Test.

Rats in TG4 (cyclosporin A and medium dose rosiglitazone) show significantly greater muscle area (i.e. lower levels of muscle necrosis) at 60 days (p=0.006) and 120 days (p=0.013) post-transplantation than those in TG1 receiving conventional treatment with cyclosporin A alone (p<0.001).

### (v) Inflammatory Cell Infiltration

The levels inflammatory cell infiltration observed in TG1 (cyclosporin A only), TG4 (cyclosporin A and medium dose rosiglitazone) and TG5 (syngenic transplant) were compared to normal heart tissue (normal being defined as tissue taken from healthy male age matched Lewis strain rats). Inflammatory cell infiltration was determined by measuring the nuclear area (as a percentage of total area).

TABLE 6
Nuclear area

| Timepoint | Treatment Group | Mean | SD | N |
|---|---|---|---|---|
| Day 120 | Normal | 4.5 | 0.83 | 30 |
| | TG5 (syngenic transplant) | 6.76 | 2.85 | 30 |
| | TG1 (cyclosporin A only) | 14.56 | 4.42 | 30 |
| | TG4 (cyclosporin A and medium dose | 7.87 | 1.78 | 30 |
| | rosiglitazone) | | | |
| Day 60 | TG5 (syngenic transplant) | 8.99 | 4.16 | 30 |
| | TG1 (cyclosporin A only) | 15.9 | 5.14 | 30 |
| | TG4 (cyclosporin A and medium dose | 8.1 | 1.22 | 30 |
| | rosiglitazone) | | | |

Samples were taken from three animals at each data point (one slide per subject and 10 fields per slide).

FIG. 5 illustrates the data graphically.

The data were analyzed using GraphPad InStat software and probability values were calculated by using Tukey-Kramer Multiple Comparison Test.

Rats in TG4 (cyclosporin A and medium dose rosiglitazone) show significantly lower nuclear areas at 60 days (p>0.001) and 120 days (p>0.001) post-transplantation than those in TG1 receiving conventional treatment with cyclosporin A alone.

### (vi) Number of Macrophages

The number of macrophages in the inflammatory cell infiltrate of rats in TG1 (cyclosporin A only), TG4 (cyclosporin A and medium dose rosiglitazone) and TG5 (syngenic transplant) was determined by counting ED-1 positive cells in immunohistochemically stained sections. The data indicates the number of positive cells per x400 field. Five fields were analysed per slide, with slides taken from three animals in each group.

TABLE 7
Number of macrophages

| Timepoint | Treatment Group | Mean | SD |
|---|---|---|---|
| Day 120 | TG5 (syngenic transplant) | 5.97 | 1.9 |
| | TG1 (cyclosporin A only) | 31.7 | 13.28 |
| | TG 4 (cyclosporin A and medium dose | 17.75 | 7.74 |
| | rosiglitazone) | | |
| Day 60 | TG5 (syngenic transplant) | 3.825 | 1.71 |
| | TG1 (cyclosporin A only) | 37.46 | 7.7 |
| | TG4 (cyclosporin A and medium dose | 13.75 | 4.55 |
| | rosiglitazone) | | |
| Day 30 | TG1 (cyclosporin A only) | 24.55 | 5.96 |
| | TG4 (cyclosporin A and medium dose | 15.03 | 4.23 |
| | rosiglitazone) | | |

FIG. 6 illustrates the data graphically.

The data were analyzed using GraphPad InStat software and probability values were calculated by using Tukey-Kramer Multiple Comparison Test.

Rats in TG4 (cyclosporin A and medium dose rosiglitazone) show a significantly lower number of macrophages at 30 days (p=0.005), 60 days (p=0.005) and 120 days (p=0.005) post-transplantation than those in TG1 receiving conventional treatment with cyclosporin A alone.

(vii) TNF-Alpha Production

Serum levels of tumour necrosis factor alpha (TNF-alpha) were measured for rats in TG1 (cyclosporin A only), TG4 (cyclosporin A and medium dose rosiglitazone) and TG5 (syngenic transplant). Serum levels of TNF-alpha were determined using an ELISA kit supplied by BioScience and are presented as pg/ml.

TABLE 8
TNF-alpha levels

| | | | | Number of |
|---|---|---|---|---|
| Timepoint | Treatment Group | Mean | SD | animals |
| Day 120 | TG5 (syngenic transplant) | 1.381 | 0.62 | 3 |
| | TG1 (cyclosporin A only) | 2.46 | 1.11 | 3 |
| | TG4 (cyclosprin A and medium | 1.69 | 0.5 | 4 |
| | dose rosiglitazone) | | | |
| Day 60 | TG5 (syngenic transplant) | 1.24 | 0.27 | 4 |
| | TG1 (cyclosporin A only) | 2 | 0.2 | 5 |
| | TG4 (cyclosporin A and medium | 0.99 | 0.1 | 2 |
| | dose rosiglitazone) | | | |
| Day 30 | TG5 (syngenic transplant) | 0.9 | 0.1 | 3 |
| | TG1 (cyclosporin A only) | 1.9 | 0.3 | 4 |
| | TG4 (cyclosporin A and medium | 2.1 | 0.78 | 4 |
| | dose rosiglitazone) | | | |

FIG. 7 illustrates the data graphically.

The data were analyzed using GraphPad InStat software and probability values were calculated by using Tukey-Kramer Multiple Comparison Test.

Rats in TG4 (cyclosporin A and medium dose rosiglitazone) show significantly lower serum levels of TNF-alpha at 60 days (p=0.005) post-transplantation than those in TG1 receiving conventional treatment with cyclosporin A alone. No significant difference was found at 120 days post-transplantation, however, this may be due to the reduced data set this late time-point where fewer animals remain.

(viii) Alloantibodies

Serum samples were collected from transplanted animals at the time of sacrifice and used as the first antibody to label thymocytes isolated from F344 rat. Serial dilution of the serum was performed (1:3, 1:10, 1:30 and 1:100). At 1:3 dilutions, the percentage of positive cells was not saturated; therefore the subsequent data was obtained and analysed at 1:3 dilutions. The FITC-conjugated anti-rat isotype specific antibody IgM was used as the secondary antibodies (Pharmingen, Calif., USA). The samples were analysed by flow cytometery (FACS Caliber, Calif. USA). Forward versus side scatter defined gating on the lymphocyte population and the results obtained were analysed using CELLQuest software.

The percentage of positive cells was used as the indication of relative antibody levels. Serum samples from 3 transplanted animals were analyzed in each experimental group.

Rats in TG1 (cyclosporin A only), TG4 (cyclosporin A and medium dose rosiglitazone) and TG5 (syngenic transplant) were measured against serum samples from syngenic transplant as a negative control. Additionally, samples were taken from animals which had been in TG1 and TG4 but had rejected their transplants. Serum was collected and frozen at the time of sacrifice (or, where appropriate, at the time of rejection).

TABLE 9
Serum IgM levels.

| | | | | Number of |
|---|---|---|---|---|
| Timepoint | Treatment Group | Mean | SD | animals |
| Day 120 | TG5 (syngenic transplant) | 9.42 | 0.54 | 3 |
| | TG1 (cyclosporin A only) | 15.61 | 1.09 | 3 |
| | TG4 (cyclosporin A and medium | 10.03 | 2.59 | 3 |
| | dose rosiglitazone) | | | |
| Day 90 | TG5 (syngenic transplant) | 13.65 | 6.92 | 3 |
| | TG1 (cyclosporin A only) | No Data | — | — |
| | TG4 (cyclosporin A and medium | 15.67 | 1.93 | 3 |
| | dose rosiglitazone) | | | |
| Day 60 | TG5 (syngenic transplant) | 9.23 | 1.06 | 3 |
| | TG1 (cyclosporin A only) | No Data | — | — |
| | TG4 (cyclosporin A and medium | 11.36 | 1.92 | 3 |
| | dose rosiglitazone) | | | |
| Rejected | TG1 (cyclosporin A only) | 19.71 | 4.6 | 3 |
| | TG4 (cyclosporin A and medium | 16.85 | 5.06 | 3 |
| | dose rosiglitazone) | | | |

FIG. 8 illustrates the data graphically.

No data is available for TG1 at 60 or 90 days.

The data were analyzed using GraphPad InStat software and probability values were calculated by using Tukey-Kramer Multiple Comparison Test.

Rats in either TG4 (cyclosporin A and medium dose rosiglitazone) or TG1 (cyclosporin alone) which have rejected the allograft demonstrate significantly higher levels of antibody than those in TG5 (syngeneic transplant).

Furthermore, it should be noted that rats in TG1 which survive to 120 days post-transplantation show significantly higher levels of antibody than those in TG5 (syngeneic transplant) (p=0.019). This finding suggests that although transplant rejection has not occurred, the recipient continues to undergo an immune response towards the graft and a risk of rejection remains.

### Conclusion

Treatment using the combination of an immunosuppressant and PPAR-gamma agonist according to the method of the invention leads to each of the key contributory elements to inflammatory tissue remodelling (namely cellular infiltration, vascular occlusion and fibrosis) being reduced. This provides evidence for the application of the method to undesirable immune responses in general, and particularly those where chronic inflammation is a major symptom.

It is clear that administration of rosiglitazone alone, following the initial phase of treatment with a combination of immunosuppressant and PPAR-gamma agonist, is sufficient to maintain immune suppression and protect the transplanted organ from rejection.

### Example 3

### Cardiac allograft rejection in rats—long term survival

### General

Transplant recipients were healthy male Lewis strain rats of 8-10 weeks age. Donor organs were harvested from healthy male F344 strain rats or Lewis strain rats of 8-10 weeks age. Animals were obtained from the Animal Resources Centre, Australia. The rats were allowed free access to food and water in a 12-hour light/12-hour dark cycled room. The experimental protocol was approved by the Committee on the Use of Live Animals in Teaching and Research, University of Hong Kong.

Rats were subject to one of three experimental protocols according to the transplant methodology applied and the medicaments administered. Six rats were used in each treatment group.

The PPAR-gamma agonist rosiglitazone was employed as rosiglitazone maleate (Avandia).

### Transplantation

### (i) Treatment Group A (TGA)—Immunosuppressant Only

Recipient rats in TGA received hearts harvested from donor F334 strain rats. Standard methods of graft harvesting and transplant were used (M. E. Russell et al., Proc. Natl. Acad. Sci. USA (1993) 90:6086-6090).

### (ii) Treatment Group B (TGB)—PPAR-Gamma Agonist Only

Rats in TGB received transplants following an identical procedure to that described for TGA.

(iii) Treatment Group C (TGC)—Immunosuppressant and Medium Dose PPAR-Gamma Agonist

Rats in TGC received transplants following an identical procedure to that described for TGA.

### Treatment Regimes

### (i) Treatment Group A—Immunosuppressant Only

Immunosuppressant (cyclosporin A) was administered by intraperitoneal injection at 20 mg/kg/day. Administration was started on the day of transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

### (ii) Treatment Group B—Medium Dose PPAR-Gamma Agonist Only

PPAR-gamma agonist (rosiglitazone) was administered orally at 5 mg/kg/day. 4 mg rosiglitazone tablets were dissolved in distilled water and the rosiglitazone solution was feed to the rats once daily by gavage, in the morning. Administration was begun 3 days prior to transplantation and was continued until 120 days after transplantation.

(iii) Treatment Group C—Immunosuppressant and Medium Dose PPAR-Gamma Agonist

Immunosuppressant (cyclosporin A) was administered by intraperitoneal injection at 20 mg/kg/day. Administration was started on the day of transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

PPAR-gamma agonist (rosiglitazone) was administered orally at 5 mg/kg/day. 4 mg rosiglitazone tablets were dissolved in distilled water and the rosiglitazone solution was feed to the rats once daily by gavage, in the morning. Administration was begun 3 days prior to transplantation and was continued until 120 days after transplantation.

### Results

Table 10 below shows the graft survival rates. Graft survival is defined as the presence of a palpable heartbeat.

TABLE 10
Graft survival time

| | | | Mean Survival |
|---|---|---|---|
| | | | Time |
| TG | Graft Survival Time | n | (days) |
| TGA | 18, 39, 47, 48, 48, >120 | 6 | 53.3 |
| TGB | 13, 13, 13, 16, 20, 20 | 6 | 15.8 |
| TGC | 35, 44, >120, >120, >120, >120 | 6 | 93.2 |

FIG. 9 shows the data as a plot of graft survival against time.

Graft survival was analysed using a chi-squared test.

Treatment using the combination of the present invention, TGC, showed significantly better survival rates than treatment according to the conventional method of TGA.

### Conclusion

The graft survival results from Example 3 indicate that the method of the invention may be expected to increase long-term graft survival rates in transplant patients. The secondary treatment phase with PPAR-gamma agonist alone was ceased on day 120, though despite the absence of medication rats survive beyond this point.

### Example 3 (Supplemental)

### Cardiac Allograft Rejection in Rats—Long Term Survival

### General

Example 3 was extended to include a number of alternative treatment groups and additional numbers of animals in certain of the treatment groups already investigated.

The general procedure was as described above.

### Transplantation

### (i) Treatment Group D (TGD)—Syngenic Graft

Recipient rats in TGD received hearts harvested from donor Lewis strain rats. Save for the donor rat strain, transplant methodology followed an identical procedure to that described for TGA.

### (ii) Treatment Group E (TGE)—Allograft Only

Rats in TGE received transplants following an identical procedure to that described for TGA.

(iii) Treatment Group F (TGF)—High Dose PPAR-Gamma Agonist Only

Rats in TGF received transplants following an identical procedure to that described for TGA.

### Treatment Regimes

### (i) Treatment Group D—Syngenic Graft

Immunosuppressant and PPAR-gamma agonist were not administered to rats in TGD.

### (ii) Treatment Group E—Allograft Only

Immunosuppressant and PPAR-gamma agonist were not administered to rats in TGE.

(iii) Treatment Group F—High Dose PPAR-Gamma Agonist Only

PPAR-gamma agonist (rosiglitazone) was administered orally at 20 mg/kg/day. 4 mg rosiglitazone tablets were dissolved in distilled water and the rosiglitazone solution was feed to the rats once daily by gavage, in the morning. Administration was begun 3 days prior to transplantation and was continued until 120 days after transplantation.

### Results

Table 10b below summarises the graft survival rates, including both the original and supplemental data. Graft survival is defined as the presence of a palpable heartbeat.

TABLE 10b
Graft Survival Time (Extended Data Set)

| | | | Mean Survival |
|---|---|---|---|
| | | | Time |
| TG | Graft Survival Time | n | (days) |
| TGA | 18, 21, 30, 32, 39, 39, 39, 44, 47, | 18 | 59.1 |
| | 48, 48, 57, 59, 64, >119*, >119*, | | |
| | >120, >120 | | |
| TGB | 13, 13, 13, 16, 20, 20 | 6 | 15.8 |
| TGC | 24, 35, 36, 43, 44, 49, 59, 60, 69, | 6 | 80.8 |
| | 76, >120, >120, >120, >120, >120, | | |
| | >120, >120, >120 | | |
| TGD | >120, >120, >120 | 3 | 120.0 |
| TGE | 10, 13, 17, 17, 18, 20 | 6 | 15.8 |
| TGF | 17, 18, 18, 20, 20, 20 | 6 | 18.8 |

*As a result of an error in timings, two animals were sacrificed after 119 days, one day earlier than the expected conclusion of the experiment

Treatment using the combination of the present invention, TGC, shows better survival rates than treatment according to the conventional method of TGA (p<0.01).

Furthermore, treatment using a PPAR-gamma agonist alone at a medium dose (TGB) or a high dose (TGF) does not show any notable difference to untreated subjects (TGE). As such, the synergistic effect of the combination of the present invention is particularly surprising.

### Example 4

### Alternate Dosage Regimes

### General

Experiments in Example 4 were performed to investigate the effects of alternative dosage levels of immunosuppressant and/or PPAR-gamma agonist.

Transplant recipients were healthy male Lewis strain rats of 8-10 weeks age. Donor organs were harvested from healthy male F344 strain rats or Lewis strain rats of 8-10 weeks age.

Animals were obtained from the Animal Resources Centre, Australia. The rats were allowed free access to food and water in a 12-hour light/12-hour dark cycled room. The experimental protocol was approved by the Committee on the Use of Live Animals in Teaching and Research, University of Hong Kong.

Rats were subject to one of three experimental protocols according to the transplant methodology applied and the medicaments administered. Six or seven rats were used in each treatment group.

The PPAR-gamma agonist rosiglitazone was employed as rosiglitazone maleate (Avandia).

### Transplantation

(i) Treatment Group I (TGI)—Cyclosporin A 20 mg/kg/day and Rosiglitazone 20 mg/kg/day

Recipient rats in TGI received hearts harvested from donor F334 strain rats. Standard methods of graft harvesting and transplant were used (M. E. Russell et al., Proc. Natl. Acad. Sci. USA (1993) 90:6086-6090).

(ii) Treatment Group II (TGII)—cyclosporin A 10 mg/kg/day and rosiglitazone 5 mg/kg/day

Rats in TGII received transplants following an identical procedure to that described for TGI.

(iii) Treatment Group III (TGIII)—Cyclosporin A 10 mg/kg/day

Rats in TGIII received transplants following an identical procedure to that described for TGI.

### Treatment Regimes

(i) Treatment Group I—Cyclosporin A 20 mg/kg/day and Rosiglitazone 20 mg/kg/day

Immunosuppressant (cyclosporin A) was administered by intraperitoneal injection at 20 mg/kg/day. Administration was started on the day of transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

PPAR-gamma agonist (rosiglitazone) was administered orally at 20 mg/kg/day. 4 mg rosiglitazone tablets were dissolved in distilled water and the rosiglitazone solution was feed to the rats once daily by gavage, in the morning. Administration was begun 3 days prior to transplantation and was continued until 120 days after transplantation.

(ii) Treatment Group II—Cyclosporin A 10 mg/kg/day and Rosiglitazone 5 mg/kg/day

PPAR-gamma agonist (rosiglitazone) was administered orally at 5 mg/kg/day. 4 mg rosiglitazone tablets were dissolved in distilled water and the rosiglitazone solution was feed to the rats once daily by gavage, in the morning. Administration was begun 3 days prior to transplantation and was continued until 120 days after transplantation.

(iii) Treatment Group III—Cyclosporin A 10 mg/kg/day

Immunosuppressant (cyclosporin A) was administered by intraperitoneal injection at 10 mg/kg/day. Administration was started on the day of transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

Table 11 below shows the graft survival rates. Graft survival is defined as the presence of a palpable heartbeat.

TABLE 11
Graft survival time

| | | | Mean Survival |
|---|---|---|---|
| | | | Time |
| TG | Graft Survival Time | n | (days) |
| TGI | 33, 37, 51, >120, >120, >120, >120 | 7 | 85.6 |
| TGII | >120, >120, >120, >120, >120, >120 | 6 | 120.0 |
| TGIII | 34, 34, 58, >120, >120, >120 | 6 | 81.0 |

The data is illustrated in FIG. 10.

Treatment using Cyclosporin A at 10 mg/kg/day in combination with rosiglitazone at 5 mg/kg/day (TGII) showed the highest graft survival rate, which is significantly improved over treatment with Cyclosporin A at 10 mg/kg/day alone (p<0.05).

### Example 5

### Persistence of Immunosuppression: Rejection of a Secondary Cardiac Allograft

### General

Transplant recipients were Lewis strain rats from which had survived for 120 days following treatment according to that described for Treatment Group C (cyclosporin A and medium dose rosiglitazone) of Example 3. Secondary transplant donor organs were harvested from healthy male F344 or DA strain rats of 8-10 weeks age.

Animals were obtained from the Animal Resources Centre, Australia. The rats were allowed free access to food and water in a 12-hour light/12-hour dark cycled room. The experimental protocol was approved by the Committee on the Use of Live Animals in Teaching and Research, University of Hong Kong.

Rats were subject to one of two experimental protocols according to the source of the donor organ utilised in the secondary transplant.

### Transplantation

Secondary transplantation was performed using the same methodology as the primary transplantation (M. E. Russell et al., Proc. Natl. Acad. Sci. USA (1993) 90:6086-6090), with the vascularised secondary graft being attached below the primary graft. Secondary transplantation was completed 120 days following the primary transplantation.

### Treatment Regimes

Rats in Example 5 did not receive either immunosuppressant or PPAR-gamma agonist during the course of the experiment.

### Results

Graft survival (as defined by the method described in Example 3) was measured for the primary and secondary grafts. Four rats received secondary grafts from F344 donors and four rats received secondary grafts from DA donors. Survival times are shown in Table 12 below.

TABLE 12
Primary and secondary transplant survival

| | Primary Transplant | Secondary Transplant |
|---|---|---|
| Treatment Group | Survival (days) | Survival (days) |
| Secondary F344 graft | 180* | 60* |
| Secondary DA graft | 161* | 5.5 |

*Experiment terminated

### Conclusion

Despite the absence of either the immunosuppressant or PPAR-gamma agonist at the time of the secondary transplant, primary transplanted organs continue to survive and secondary transplanted organs from the same strain are not rejected during the course of the experiment (Secondary F344 graft). This finding contrasts with those animals receiving secondary transplants from a different strain (Secondary DA graft) where the secondary transplant was rejected within a number of days.

These data indicate that the method of the present invention results in the establishment of a donor specific tolerance while not preventing immune responses against other alloantigens.

Extended therapy using immunosuppressive agents (or the combination of an immunosuppressive agent and PPAR-gamma agonist according to the present invention) is not required. This finding has significant implications for field of organ and tissue transplant, both in reducing the costs associated with long-term treatment using conventional methods and the side effects suffered by transplant recipients who require conventional long-term treatment.

A further implication is that a donor specific tolerances could be developed using a sacrificial tissue graft. While this is not generally applicable due to the source of the majority of donor organs, potential recipients with a known organ donor (such as a family member) may benefit.

The development of a donor specific tolerance may be expected to be of great value in the treatment of autoimmune disorders. A patient may regain tolerance to the auto-antigens which are at the root of the disorder.

### Example 5 (Supplemental)

### Persistence of Immunosuppression: Rejection of a Secondary Cardiac Allograft

### General

Example 5 was extended to include a number of alternative treatment groups.

The general procedure was as described above i.e. rats receiving different treatment regimes were given an initial transplant from F344 strain animals and those surviving after 120 days were then given a secondary graft from either F344 or DA strain animals.

### Treatment Regimes

(i) Treatment Group W—Cyclosporin A 10 mg/kg/day

Immunosuppressant (cyclosporin A) was administered by intraperitoneal injection at 10 mg/kg/day. Administration was started on the day of the primary transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

(ii) Treatment Group X—Cyclosporin A 20 mg/kg/day

Immunosuppressant (cyclosporin A) was administered by intraperitoneal injection at 10 mg/kg/day. Administration was started on the day of the primary transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

(iii) Treatment Group Y—Cyclosporin A 10 mg/kg/day and Rosiglitazone 5 mg/kg/day

Immunosuppressant (cyclosporin A) was administered by intraperitoneal injection at 10 mg/kg/day. Administration was started on the day of the primary transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

PPAR-gamma agonist (rosiglitazone) was administered orally at 5 mg/kg/day. 4 mg rosiglitazone tablets were dissolved in distilled water and the rosiglitazone solution was feed to the rats once daily by gavage, in the morning. Administration was begun 3 days prior to primary transplantation and was continued until 120 days after primary transplantation.

(iv) Treatment Group Z—Cyclosporin A 20 mg/kg/day and Rosiglitazone 5 mg/kg/day

Immunosuppressant (cyclosporin A) was administered by intraperitoneal injection at 20 mg/kg/day. Administration was started on the day of the primary transplant, following completion of surgical procedures, and was continued for a total of 14 days.

A 16.6 mg/ml solution of cyclosporin A for administration was prepared daily by dilution of concentrated cyclosporin A (Sandimmun, 50 mg/ml, Novartis) with saline (0.9% NaCl). One injection of cyclosporin A was given each morning.

PPAR-gamma agonist (rosiglitazone) was administered orally at 5 mg/kg/day. 4 mg rosiglitazone tablets were dissolved in distilled water and the rosiglitazone solution was feed to the rats once daily by gavage, in the morning. Administration was begun 3 days prior to primary transplantation and was continued until 120 days after primary transplantation.

### Results

Graft survival (i.e. the presence of a palpable heart beat) was measured for the primary and secondary grafts. Secondary graft survival times are shown in Table 12b below.

TABLE 12b
Primary and secondary transplant survival

| | | | | Mean Secondary Graft |
|---|---|---|---|---|
| Treatment | Secondary | | Secondary Graft | Survival Time |
| Group | Graft | n | Survival Time | (days) |
| TGW | F344 | 2 | >60, >60 | 60 |
| | DA | 1 | 6 | 6 |
| TGX | F344 | 1 | >60 | 60 |
| TGY | F344 | 3 | >60, >60, >60 | 60 |
| | DA | 2 | 7, 7 | 7 |
| TGZ | F344 | 2 | >60, >60 | 60 |
| | DA | 2 | 7, 7 | 7 |

All primary grafts survived until the end of the experiment (i.e. 180 days in total).

Histology data at the end of the experiment is shown in FIG. 11: Sections A to C are from secondary grafts from treatment group TGX (i.e. cyclosporin A 20 mg/kg/day, 60 days post transplant); Sections D to F are from secondary grafts from treatment group TGZ (i.e. cyclosporin A 20 mg/kg/day and rosiglitazone 5 mg/kg/day, 60 days post transplant); Sections G to I are from the primary grafts from treatment group TGX (i.e. cyclosporin A 20 mg/kg/day, 180 days post transplant); and Sections J to L are from the primary grafts from treatment group TGZ (i.e. cyclosporin A 20 mg/kg/day and rosiglitazone 5 mg/kg/day, 180 days post transplant). As can be seen from the slides, all tissue samples show comparable levels of remodelling.

### Conclusion

Despite the absence of either the immunosuppressant or PPAR-gamma agonist at the time of the secondary transplant, secondary transplanted organs from the same strain are not rejected during the course of the experiment (Secondary graft F344, for all four treatment groups). This finding contrasts with those animals receiving secondary transplants from a different strain (Secondary graft DA, for all treatment groups with subjects) where the secondary transplant was rejected within a number of days.

These data indicate that the method of the present invention results in the establishment of a donor specific tolerance while not preventing immune responses against other alloantigens.

A degree of caution should be utilised in considering the data in Table 12b. It must be recalled that, although the data is comparable between treatment regimes, only animals surviving to 120 days after the initial graft continue through to the second stage. As such, the data is not incompatible with earlier findings showing that single graft survival is higher using treatment according to the invention as opposed to conventional treatment. Rather, the data in this supplemental experiment suggests that irrespective of treatment regime, the key factor in graft survival is the development of a donor specific tolerance and a similar tolerance can develop under different treatment regimes, but is facilitated by the present invention.

Example 2 previously found a clear difference in the extent of tissue remodelling at the 120 day time point (the point at which treatment in the present example was ceased) between animals treated according to the present invention and those receiving conventional treatment. The absence of a notable difference in the extent of tissue remodelling observed in primary grafts at the end of the present experiment indirectly suggests that continued administration of PPAR-gamma agonist may be desirable.

All references including patent and patent applications referred to in this application are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

1. A method for the treatment or prevention of an undesirable immune response, comprising the simultaneous administration of an immunosuppressant and a PPAR-gamma agonist.

2. A method according to claim 1, comprising:
(a) an initial treatment phase comprising the simultaneous administration of an immunosuppressant and a PPAR-gamma agonist;
(b) a subsequent treatment phase comprising the administration of a PPAR-gamma agonist without the immunosuppressant of phase (a).

3. A method according to claim 1, wherein, comprising:
(a) an initial treatment phase comprising the simultaneous administration of an immunosuppressant and a PPAR-gamma agonist;
(b) a subsequent treatment phase comprising the administration of a PPAR-gamma agonist without an immunosuppressant.

4. A method according to claim 1, wherein, the immunosuppressant is utilized at a level which is sub-therapeutic in the absence of the PPAR-gamma agonist.

5. A method according to claim 1, wherein, the PPAR-gamma agonist is farglitazar.

6. A method according to claim 1, wherein, the PPAR-gamma agonist is a thiazolidinedione.

7. A method according to claim 6, wherein the thiazolidinedione is pioglitazone or rosiglitazone.

8. A method according to claim 7, wherein the thiazolidinedione is rosiglitazone.

9. A method according to claim 1, wherein, the immunosuppressant is not a cyclosporin.

10. A method according to claim 9, wherein the immunosuppressant is not cyclosporin A.

11. A method according to claim 1, wherein, the immunosuppressant is a macrolide.

12. A method according to claim 11, wherein the immunosuppressant is selected from pimecrolimus, tacrolimus and sirolimus.

13. A method according to claim 12, wherein the immunosuppressant is tacrolimus.

14. A method according to claim 1, wherein the immunosuppressant is cyclosporin A.

15. A method according to claim 1, wherein, the immunosuppressant is a biological immunosuppressant.

16. A method according to claim 1, wherein, the immune response comprises chronic inflammation.

17. A method according to claim 1, wherein, the immune response comprises tissue remodeling.

18. A method according to claim 17, wherein the immune response comprises a cellular infiltration.

19. A method according to claim 17, wherein the immune response comprises vascular occlusion.

20. A method according to claim 17, wherein the immune response comprises fibrosis.

21. A method according to claim 1, wherein, the undesirable immune response is a transplant rejection

22. A method according to claim 21, wherein the transplant is a solid organ transplant.

23. A method according to claim 22, wherein the solid organ transplant is selected from the group consisting of: kidney, liver, heart, lung, small bowel and limb transplants.

24. A method according to claim 21, wherein the transplant is a bone marrow transplant.

25. A method according to claim 21, wherein the initial treatment phase (a) commences following transplantation.

26. A method according to claim 25, wherein the initial treatment phase (a) is preceded by a pre-treatment phase comprising the administration of a PPAR-gamma agonist without an immunosuppressant.

27. A method according to claim 1, wherein, the undesirable immune response is an autoimmune disorder.

28. A method according to claim 27, wherein the autoimmune disorder is selected from the group consisting of: rheumatoid arthritis, psoriasis and systemic lupus erythematosus.

29. A method according to claim 28, wherein the autoimmune disorder is psoriasis.

30. A method according to claim 28, wherein the autoimmune disorder is rheumatoid arthritis.

31. A method according to claim 28, wherein the autoimmune disorder is systemic lupus erythematosus.

32. A method according to claim 27, wherein the undesirable immune response is a disorder with an autoimmune component.

33. A method according to claim 32, wherein the disorder with an autoimmune component is selected from the group consisting of: inflammatory bowel disease (including ulcerative colitis and Crohn's disease), Hashimoto's thyroiditis, pernicious anemia, Addison's disease, type I diabetes, systemic dermatomyositis, Sjogren's syndrome, multiple sclerosis, myasthenia gravis, Reiter's syndrome and Grave's disease.

34. A method according to claim 32, wherein the disorder with an autoimmune component is inflammatory bowel disease.

35. A method according to claim 32, wherein the disorder with an autoimmune component is multiple sclerosis.

36. A method according to claim 27, wherein the undesirable immune response is a disorder with an inflammatory component and which may or may not be autoimmune related.

37. A method according to claim 36, wherein the disorder with an with an inflammatory component and which may or may not be autoimmune related is atherosclerosis.

38. A pharmaceutical composition comprising an immunosuppressant and a PPAR-gamma agonist.

39. A pharmaceutical composition according to claim 38, additionally comprising a pharmaceutically acceptable diluent or carrier.

40-60. (canceled)
